(19) United States
(12) Patent Application Publication
(10) Pub. No.: US 2022/0287873 A1
(43) Pub. Date: Sep. 15, 2022

(54) BODY TEMPERATURE REGULATION

(71) Applicant: Aleksandr TASLAGYAN, South Lake Tahoe, CA (US)

(72) Inventor: Aleksandr TASLAGYAN, South Lake Tahoe, CA (US)

(21) Appl. No.: 17/693,115

(22) Filed: Mar. 11, 2022

Related U.S. Application Data
(60) Provisional application No. 63/160,869, filed on Mar. 14, 2021.

(51) Int. Cl. A61F 7/00

(52) U.S. Cl. CPC A61F 7/007; A61F 2007/0011; A61F 2007/0018; A61F 2007/0035; A61F 2007/0041; A61F 2007/008; A61F 2007/0044; A61F 2007/0064

(57) ABSTRACT

A body temperature regulation device includes a wearable article, an enclosure on the article and defining an air path through the body temperature regulation device, a heat exchange system within the enclosure and extending through the article to expose a surface of the heat exchange system at an inner surface of the article, and a blower attached to the article and positioned to direct an air flow through the heat exchange system. The article may be worn on a human limb with the surface of the heat exchanger contacting a wearer at a body location where blood flows close to skin. Heat flows through the heat exchanger between the wearer and air flow to heat or cool the blood flows and to regulate the body temperature of the wearer.

### REFERENCE TO RELATED APPLICATIONS

This patent document claims benefit of the earlier filing date of U.S. provisional Pat. App. No. 63/160,869, filed Mar. 14, 2021, which is hereby incorporated by reference in its entirety.

### BACKGROUND OF THE INVENTION

Cells in a human body need to remain in a specific temperature range to work properly. To keep cells at a proper temperature, the human body has a thermoregulations system that reacts to temperature receptors located throughout the body and makes physiological adjustments to try to maintain a constant core temperature. A person's brain, skin, hormones, sweat glands and blood vessels are all part of the system that helps regulate body temperature. Normally, the thermoregulations system in a healthy human body can maintain a suitable body temperature in a wide range of environments. Very cold, hot, or humid environments may, however, strain the ability of even a healthy body to maintain proper body temperature. Additionally, some people have body temperature regulation problems. High body temperature, i.e., hyperthermia or fever, and very low body temperature, i.e., hypothermia, are both dangerous.

### SUMMARY OF THE INVENTION

In accordance with an aspect of the invention, body temperature regulation devices and processes allow conductive cooling (or heating) of a targeted body locations to cool (or heat) the blood passing through the location. Cooled (or heated) blood travels to the other parts of the body lowering the overall body temperature. Such devices and processes assist a person in regulating their body temperature by regulating the temperature of just one or more particular parts of the body and using the bodies circulatory system and blood as a working heat exchanging fluid passing through the part of the body being cooled or warmed to assist temperature regulation though out the entire body.

One example of a body temperature regulation device may be based on thermoelectric modules, which are capable of moving heat between two surfaces of the module in a direction that depends on the direction of electrical current through the module. A worn portion of the body temperature regulation device may include one or more of a thermal conductor, a thermoelectric module, a heat exchanger, and a blower. A wearable article such as a band, a strap, or article of clothing holds the device the thermal conductor in contact with a part of the human body having high blood flows close to the skin, e.g., in an area with a high density of blood vessels close to the skin. For cooling, heat from the skin flows through the thermal conductor to the thermal electric module, where the thermoelectric module drives the heat into the heat exchange and an air flow that the blower drives through the heat exchange removes the heat from the body temperature regulation device. For heating, an air flow that the blower drives through the heat exchange may keep the heat exchanger at an ambient temperature, and the thermoelectric module drives heat from the heat exchanger through the thermal conductor to the skin and blood of the wearer of the body temperature regulation device. A battery or an external power source may supply power to the thermoelectric module and the blower for heating or cooling.

A body temperature regulation system in accordance with one example of the present disclosure may include one or more body temperature regulation devices worn under clothes, and one or more of these devices may be attached to tubing or other duct system to bring environmental, heated, or cooled air into the under-the-clothes device and into a heat exchanger inside the device. The heat exchanger transfers heat to or from the air flow before the air flow leaves the heat exchanger and returns to the environment. In one example, a device worn under clothing that takes air from the environment through a tube or duct and then releases the air under the clothing, through one or more air vents on an enclosure of the device.

Another example of a body temperature regulation device is based on a thermal reservoir that may be heated or cooled before a person wears the body temperature regulation device. Again, a wearable article such as a band, a strap, or article of clothing holds the device so that a thermal conductor is in contact with a part of the human body having high blood flows close to the skin, e.g., in an area with a high density of blood vessels close to the skin, heat may flow between the thermal reservoir and the contacted body part through the thermal conductor in a direction that depends on whether the thermal reservoir is hotter or colder that the body part.

Yet another example of a body temperature regulation device is based on a heating element. Again, a wearable article such as a band, a strap, or article of clothing holds the device so that a thermal conductor is in contact with a part of the human body having high blood flows close to the skin, e.g., in an area with a high density of blood vessels close to the skin. With this example, heat may flow from the heating element through the thermal conductor to the contacted body part and blood circulation through the contacted body part heat the wearer.

### BRIEF DESCRIPTION OF THE DRAWINGS

FIG. 1 is a block diagram of a body temperature regulation system in accordance with an example of the present disclosure.

FIG. 2 illustrates use of a body temperature regulation system in accordance with an example of the present disclosure including multiple body temperature regulation devices that a person wears at locations having high blood flows near the skin.

FIG. 3-1 shows perspective view of a body temperature regulation device in accordance with an example of the present disclosure.

FIG. 3-2 shows an exploded view of the body temperature regulation device of FIG. 3.

FIG. 4 is a block diagram of a body temperature regulation device in accordance with an example of the present disclosure employing a thermal reservoir.

FIG. 5 is a block diagram of a body temperature regulation device in accordance with an example of the present disclosure employing a heater.

FIG. 6 shows a perspective view of a body temperature regulation device that does not require air flow for operation.

The drawings illustrate examples for the purpose of explanation and are not of the invention itself. Use of the same reference symbols in different figures indicates similar or identical items.

### DETAILED DESCRIPTION

A body temperature regulation device in accordance with an example of the present disclosure warms or cools a part of a body where blood flow acts as a natural heat exchanging fluid to pass through the area being warmed or cooled and remove heat from or add heat to the entire body. The body temperature regulation device may include a heat exchanger, a power source, and venting. The body temperature regulation device may be used as part of a body regulation system that may remove or add body heat at one or more locations on a user's body. A body temperature regulation system may employ a thermoelectric module and an air exchanger, a high-capacity thermal reservoir, or a resistive heater to provide a heat source or sink for heating or cooling or may employ the surrounding environment as a source or sink for heat.

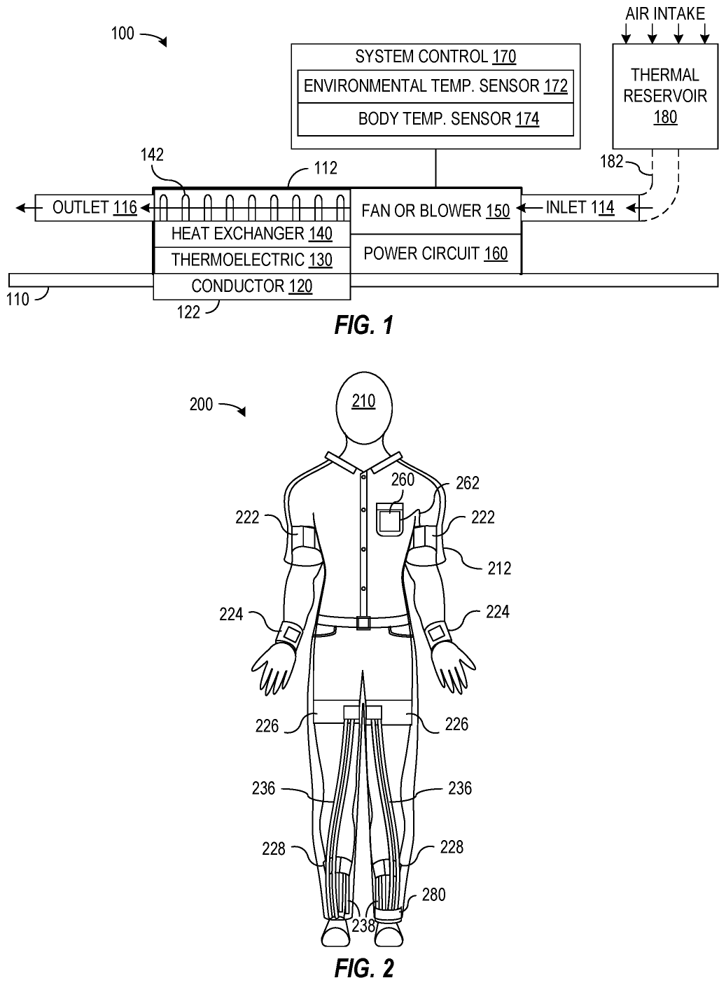

FIG. 1 shows a block diagram of a body temperature regulation device 100 in accordance with an example of the present disclosure. Body regulation device 100 is wearable on a human body and includes a wearable article 110 such as a band, strap, harness, or an article of clothing that may be worn. Wearable article 110 is particularly adapted to be worn at a body location on a body part where blood vessels or arteries are near the skin surface. For example, wearable article 110 may be worn around any part of the body, including limbs, the neck, or the head and particularly around a wrist, around an upper arm, on an ankle, around an upper thigh where larger blood vessels pass near the skin.

Body temperature regulation device 100 may be worn on bare skin or on top of the clothing. Device 100 may particularly be worn so that thermal conductor 120, which may be embedded in or otherwise pass through a portion of wearable article 110, has a surface 122 contacts an area of skin or clothing near the desired blood vessels and blood flows. Thermal conductor 120 may include a metal plate or block of other thermal conductive material that is mounted on wearable article 110 so that surface 122 of thermal conductor 120 is exposed to contact the skin or clothing of the wearer.

Wearable article 110 further defines an enclosure 112 that may house components of body temperature regulation device 100. Enclosure 112 may be made of different materials, shapes, can be flexible, rigid and or foldable. Enclosure 112 may contain one or more of a thermoelectric module 130, a heat exchanger 140, an air movement system 150, a power circuit 160, and a system control module 170. Power circuit 160, which may be under the control of system control module 170, provides power to the heat exchange module 130 or thermoelectric module 140 and may control when device 100 provides heating or cooling. Power circuit 160 or system control system 170 may include a battery to power thermoelectric module 130 and air movement system 150. In one configuration, the battery and all or parts of power circuit 160 and system control system 170 are in enclosure 112. In an alternative configuration, the batter and all or parts of power circuit 160 and system control system 170 are external to wearable article 110, e.g., kept in a pocket of clothing that the user wears and connected to electrical modules 130 and 150 through wires.

Thermoelectric module 130 includes one or more semiconductor heating or cooling units that use the Peltier effect to create a heat flow between top and bottom surfaces of module 130. Power circuit 160 can control the direction of the heat flow by controlling the direction of a current applied to thermoelectric module 130. In one current configuration, thermoelectric module 130 warms a wearer by moving heat from heat exchanger 140 to thermal conductor 120, and thermal conductor 120 conducts heat to the wearer of device 100. In an opposite current configuration, thermoelectric module 130 cools the wearer by moving heat from thermal conductor 120 to heat exchanger 140, and thermal conductor 120 removes heat from the wearer of device 100. In another configuration. In some implementations, conductor 120 may not be required, and thermoelectric module 130 extend through wearable article 110 and directly contact the skin or clothing of the wearer.

Heat exchanger 130 in the example of FIG. 1 works with air movement system 150. Air movement system 150 may be or include a fan or a blower or other system capable of directing an air flow through heat exchanger 140. Heat exchanger 140 may be made of metal or other thermally conductive material and may have fins or other structures 142 that are in the path of air flow that air movement system 150 drives. Heat exchanger 140 may further be affixed on wearable article 110 so that a bottom surface of heat exchanger contacts a top surface of thermoelectric module 130. During body heating, heat from the air flow passing through heat exchanger 140 warms heat exchanger 140, and thermoelectric module 130 moves heat from heat exchanger 140 through thermal conductor 120 to the wearer of device 100. During body cooling, thermoelectric module 130 moves heat from thermal conductor 120 and the body of the wearer of device 100 into heat exchanger 140 and the air flow through heat exchanger 140 removes heat from heat exchanger 140 and carries the removed heat out of an outlet vent 116 in housing 112.

The air flow through heat exchanger may be drawn from the surrounding environment through an inlet vent 114 into housing 112 and be exhausted back to the surrounding environment through an outlet vent 116 in enclosure 112. As described further below, air inlet vent 114 or air outlet vent 115 may be connected tubing or ducts, for example, so that air may be drawn from or exhausted to the environment outside clothing. If device 100 is covered with the clothing, for example, tubing or other duct structure may convey air from a location not covered by clothing to inlet vent 114. Tubing or other ducting structure may similarly convey air from outlet vent 116 to a location not covered by clothing, but outlet vent 116 may exhaust air flow inside clothing.

Air flow into enclosure 112 may be directly from the environment surrounding the wearer or may first flow through a thermal reservoir 180. Thermal reservoir 180 may, for example, be a bag or other container containing one or more blocks, pieces, or particles of material having a high specific heat. Thermal reservoir 180 may, for example, one or more reusable hot and cold gel ice packs or beads or other pieces of a material such as sodium polyacrylate or other material having a high specific heat. The material in thermal reservoir 180 may be heated to a temperature higher than the temperature of the surrounding environment if warming or heat flow to the wearer of device 100 is desired or may be cooled to a temperature lower than the surrounding environment if cooling or heat flow from the wearer of device 100 is desired. Thermal reservoir 180 may additionally or alternatively include a powered heating or cooling system, e.g., a thermoelectric module or resistive heater, also worn be the wearer of device 100. Thermal reservoir 180 may be relatively large and may be located outside of enclosure. For example, thermal reservoir 180 include a pouch or bag worn on a belt of the wearer of device 100 and may be connected to enclosure 112 through a tube or other air duct structure 182. During uses, air drawn into thermal reservoir 180 may flow around the pieces of high specific heat mater and be warmed or cooled before flowing through duct structure 162 to enclosure 112. In enclosure 112, blower 150 may directed the air from thermal reservoir 180 through heat exchanger 140 to provide additional heat flow to or from the body of the wearer of device 100.

System control module 170 is optional and may be used to control operation of thermoelectric module 140, air movement system 150, power circuit 160, or thermal reservoir 180. For example, system control 170 may use a body temperature sensor 174 to sense the body temperature of the wearer of device 100 and based on the body temperature may determine whether heating, cooling, or no change of body temperature is desirable. System control module 170 may also use an environmental temperature sensor 174 to measure the temperature of the environment or other source of air. Based on the air temperature, system control module 170 may determine whether the available air is suited for the desired heat flow, e.g., an environmental temperature lower than the body temperature when cooling is desired. System control 170 may then turn on heat exchange module 130, thermal electric module 140, or heating or cooling of thermal reservoir 180. System control 170 may shut off device 100 when neither heating or cooling is desired or when the available air sources are not suited for the desired heating or cooling.

System control 170 may provide an interface that allows the wearer to turn device 100 on or off or to switch device 100 between different operating modes, e.g., heating or cooling.

FIG. 2 shows a body temperature regulation system 200 in which a wearer 210 wears multiple body temperature regulation devices 222, 224, 226, and 228. Each body temperature regulation device 222, 224, 226, or 228 may have the same structure as described above with reference to FIG. 1 or alternative structures as described below. In some configurations, body temperature regulation devices 222, 224, 226, and 228 may share some resources or structural elements. For example, two or more of devices 222, 224, 226, and 228 may share a control system, e.g., control system 170 of FIG. 1, that operates the devices based on temperature measurements or on selections that wearer 210 may make. In another example, two or more of devices 222, 224, 226, and 228 may share a thermal reservoir 280, such as describe with reference to thermal reservoir 180 of FIG. 1, that heats or cools the air taken from the environment surrounding wearer 210. Devices 222, 224, 226, and 228 may also share an external power source or battery 260 having wires 262 that connects to one or more devices 222, 224, 226, 228. Wearer 210 may keep share or external resources such as a control system, battery 260, or thermal reservoir 280 in a pocket or may attach such resources to clothing or a belt, and such resources may include wearable articles allow the resources to be worn.

Body temperature regulation devices 222, 224, 226, and 228 are worn in different locations on body parts of wearer 210. Each device 222 may be worn on an upper bicep of wearer 210 and oriented so that the heat conductive surface of the device 222 directly contacts skin under the arm of wearer 210. Each device 224 may be worn on a wrist of wearer 210 and oriented so that the heat conductive surface of the device 222 directly contacts skin on the underside of the wrist of wearer 210. Each device 226 may be worn on an upper thigh of wearer 210 and oriented so that the heat conductive surface of the device 226 directly contacts skin on the inner thigh of wearer 210. Each device 228 may be worn on an ankle of wearer 210 and oriented so that the heat conductive surface of the device 228 directly contacts skin adjacent to veins or arteries in the ankle. Some of devices 224 may be worn outside clothing 212 of wearer 210 and may directly draw air from the surrounding environment and directly exhaust (after heat exchange) back to the surrounding environment. Some devices 222 worn under clothing may also directly draw air from and directly exhaust air to the surrounding environment. Some of devices 226 and 228 may be worn under clothing 212 of wearer 210 and may draw air from the surrounding environment through a tubing or duct system 236 o4 238 and may exhaust (after heat exchange) back to the surrounding environment through the duct system 236 or 238 or directly the clothing 222.

FIGS. 3-1 and 3-2 show a body temperature regulation device 300 in accordance with another example of the present disclosure. Body temperature regulation device 300 includes an adjustable band 310 that may be worn on a limb or a human user. Band 310 may be a flexible strip with a clasp or hook-and-loop fastener that allows band 310 to be wrapped around a limb and secured.

A shell 312 of a resilient material such as plastic or metal is mounted on band 310 and defines the enclosure containing modules of body temperature regulation device 330. Shell 312 further defines an air path through body temperature regulation device 330 and has one or more air inlets 314 and one or more air outlets 316. As described above, air ducts may be attached to inlets 314 or exhaust 316, particularly when body temperature regulation device 300 is worn under clothing or is used with an external thermal reservoir.

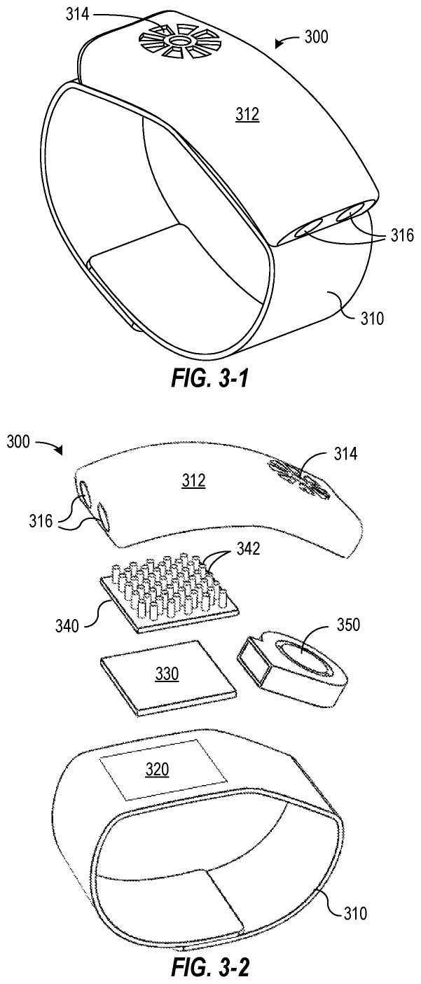

FIG. 3-2 shows an exploded view of body temperature regulation device 300. As shown, body temperature regulation device 300 includes a thermal conductor 320 that extends through band 310, a thermoelectric module, 330 on thermal conductor 320, and a heat exchanger 340 on thermoelectric module 330. A blower 350, which may be powered by a battery (not shown) is mounted on band 310 and positioned to draw air in through inlet 314 and to direct air flow through fins or projections 342 on a heat exchanger 340.

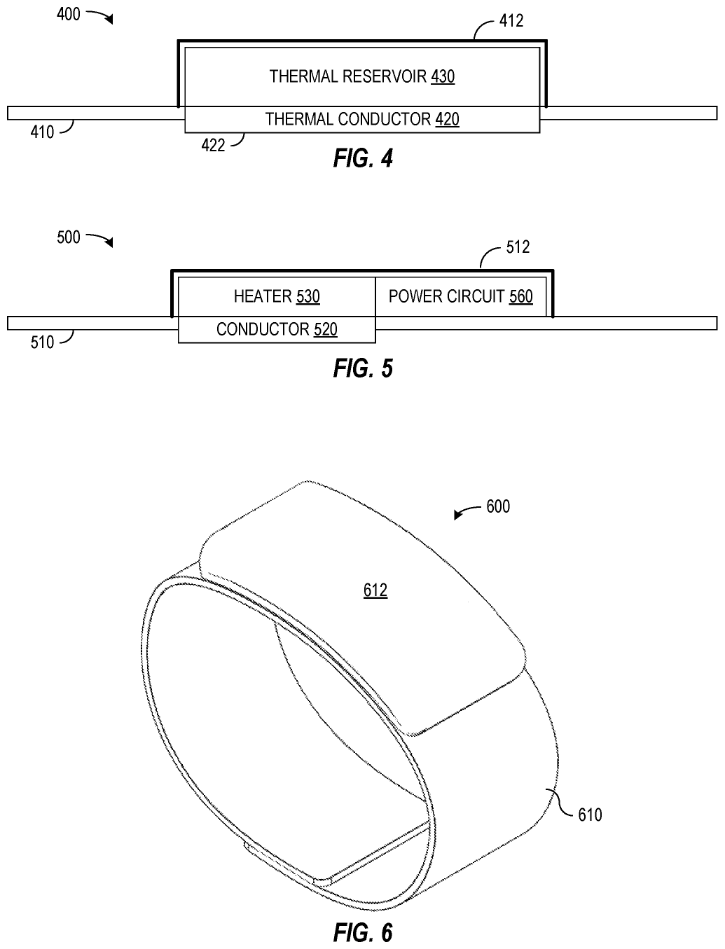

FIG. 4 is a block diagram of a body temperature regulation device 400 in accordance with an example using a thermal reservoir 430 for heating or cooling. Device 400 includes an wearable article 410 that may be worn. In general, wearable article 410 may include a band or a harness and may be substantially identical to wearable article 110 described above. Wearable article 410 for device 400 further includes a housing 412 that encloses a thermal reservoir 430. In some examples, housing removably snaps onto or affixes to a fixture on band 410 or may open to permit a user to remove or replace thermal reservoir 530. In other examples, housing 412 may permanently attach to band 410 or be difficult to open so that thermal reservoir 430, and thermal reservoir 430 is not intended to be removable from device 400.

Thermal reservoir 430 may include one or more blocks or pieces of a high specific heat material, e.g., gel ice or sodium polyacrylate, capable of holding a relatively large amount of heat. Thermal reservoir 430 may include a bag of high thermal capacity material that can take various forms and shapes of enclosures may shaped to fit various parts of the body, e.g., hands, wrists, arms, legs, ankles, feet, and torso. For use of body temperature regulation device 400, thermal reservoir 430 or the entirety of device 400 may be cooled, e.g., placed in a freezer, or heated, e.g., on a stove or oven, depending on whether heating or cooling of the wearer is desired. In the illustrated example, device 400 uses thermal conductor 420 to conduct heat from thermal reservoir 430 to the wearer for heating or conduct from the wearer to thermal reservoir 430 for cooling, and the size and thickness of thermal conductor 420 may be selected to control the rate of heat flow. Alternatively, thermal conductor 420 may be eliminated from device 400, and thermal reservoir 430 may directly contact the skin or clothing of the wearer.

FIG. 5 is a block diagram of a body temperature regulation device 500 in accordance with an example using a resistive or Ohmic heater 530 for heating. Device 500 includes an wearable article 510 that may be worn and may include a band, a harness, or any other wearable system as described above. Wearable article 510 for device 500 further includes a housing 512 that encloses resistive heater 530 and a power circuit 560. Power circuit 560 may, for example, include battery or other power source and a switch or thermostat that turns resistive heater 530 on and off. In the illustrated example, device 500 uses a thermal conductor 520 to conduct heat from resistive heater 530 to the wearer for heating.

Body temperature regulation devices 400 and 500 of FIGS. 4 and 5 do not require venting or air flows. Accordingly, examples of devices 400 or 500 may take the form of a wearable device 600 that lacks air vents as shown in FIG. 6. Wearable device 600 includes a band 610 that may be worn on a body part, and a housing 612 on band 610 may be positioned to provide heating or cooling at a body location that is adjacent to significant blood flows carried by blood vessels at the location.

Although particular implementations have been disclosed, these implementations are only examples and should not be taken as limitations. Various adaptations and combinations of features of the implementations disclosed are within the scope of the following claims.

What is claimed is:

1. A body temperature regulation device comprising:
a wearable article;
a heat conductor extending through the wearable article; and
a heat source/sink coupled to direct heat flow through the heat conductor.

2. The device of claim 1, wherein the wearable article, when worn, positions the heat conductor adjacent to blood vessels to regulate a local temperature of circulating blood, the body temperature regulation device using the blood as heat exchange liquid to regulate core body temperature.

3. The device of claim 1, wherein the heat source/sink comprises a thermoelectric module having a bottom face on the heat conductor, the thermoelectric module driving the heat flow through the heat conductor to a wearer of the wearable article in response to application of an electrical current through the thermoelectric module in a first direction, the thermoelectric module driving the heat flow through the heat conductor away from the wearer of the wearable article in response to application of an electrical current through the thermoelectric module in a second direction that is opposite to the first direction.

4. The device of claim 3, further comprising:
a heat exchanger on a top face of the heat conductor; and
an air movement system attached to the wearable article and positioned to direct an air flow through the heat exchanger.

5. The device of claim 4, further comprising a housing attached to the wearable article and defining an air path from an inlet in the housing, through the air movement system and the heat exchanger, and out of an outlet in the housing.

6. The device of claim 5, further comprising an air duct coupled to at least one of the inlet and the outlet of the housing, the air duct providing air flow between a first location where the body temperature regulation device is worn under clothing to a second location outside the clothing.

7. The device of claim 1, wherein the heat source/sink comprises a thermal reservoir in contact with the thermal conductor.

8. The device of claim 1, wherein the heat source/sink comprises a resistive heating element in contact with the thermal conductor.

9. The device of claim 1, wherein the wearable article is configured to be worn around one of a limb, a torso, a neck, or a head of a wearer of the device.

10. The device of claim 1, further comprising:
a housing attached to the wearable article and containing the heat source/sink; and
a battery inside the housing and powering the heat source/sink.

11. The device of claim 1, further comprising:
a housing attached to the wearable article and containing the heat source/sink;
a battery outside the housing; and
wires connecting the battery to the heat source/sink.

12. The device of claim 1, wherein the thermal conductor extends through a location on the wearable article such that, when a wearer wears the body temperature regulation device, a face of the thermal conductor is adjacent to a body part of having a concentration of blood vessels.

13. The device of claim 12, wherein the part of the body part is selected from the group consisting of an inner wrist, an arm pit, an inner thigh, an ankle, and a neck.

14. A body temperature regulation device comprising:
a wearable article configured be worn by a person;
a thermoelectric module mounted on the wearable article and having a first surface position to move through the surface, heat to or from the person wearing the wearable article; and
a heat exchanger on a second surface of the thermoelectric module.

15. The device of claim 14, wherein the wearable article positions the heat conductor adjacent to blood vessels to regulate a local temperature of circulating blood, the device using the blood as heat exchange liquid to regulate core body temperature.

16. The device of claim 14, further comprising a blower mounted on the wearable article and positioned to drive an air flow through the heat exchanger.

17. The device of claim 14, further comprising a heat conductor positioned on the wearable article to be between the thermoelectric module and the person wearing the wearable article, the thermoelectric module moving the heat to or from the person through the thermal conductor.

18. A process for regulating body heat, comprising:
wearing a device on a body part;
positioning a heat source/sink in a location of the body part having of blood vessels adjacent to skin; and
the heat source/sink heating/cooling blood passing through the blood vessels at the location, such that blood acts as heat exchanging fluid within a human body and adjust a core temperature of the human body.

19. The process of claim 18, wherein the heat source/sink comprises a thermoelectric module that directs heat to the blood when an electrical current applied to the thermoelectric module has a first direction that directs heat flow away from the blood when the electrical current applied to the thermoelectric module has a second direction opposite to the first direction.

20. The process of claim 18, further comprises the heat source/sink comprises a thermal reservoir, and the process further comprise heating or cooling a thermal reservoir before using the thermal reservoir in the device.
